(19) United States
(12) Patent Application Publication
(10) Pub. No.: US 2025/0176540 A1
(43) Pub. Date: Jun. 5, 2025

(54) ANTIMICROBIAL FORMULATIONS AND THEIR METHODS FOR USE

(71) Applicant: Kevin J. Seifert, Osterville, MA (US)

(72) Inventor: Kevin J. Seifert, Osterville, MA (US)

(21) Appl. No.: 19/044,405

(22) Filed: Feb. 3, 2025

Related U.S. Application Data
(63) Continuation of application No. PCT/US2023/028189, filed on Jul. 20, 2023.

(51) Int. Cl. A01N 33/08; A01N 25/04; A01N 31/02

(52) U.S. Cl. CPC A01N 33/08; A01N 25/04; A01N 31/02

(57) ABSTRACT

Antimicrobial formulations are provided for use in gels, fluids, cleaning materials, such as medical swabs and wipes, and the like and methods for decreasing the risk of bacterial colonization and biofilm on devices that in-dwell, penetrate and/or navigate in the body. An antibiotic gel formulation is provided that comprises ethylenediaminetetraacetic acid (EDTA) in about 1 percent to about 6 percent by weight. The formulations described herein may be particularly beneficial in both the prevention and treatment of biofilms found within, on, or near in-dwelling medical devices, as well as biofilms found on, or within, body tissue, such as abiotic (i.e., wound dressing) and biotic (i.e., wound bed) environments.

### CROSS-REFERENCE TO RELATED APPLICATIONS

This application is a continuation of International Application No. PCT/US2023/28189 filed Jul. 20, 2023, which claims the benefit of U.S. Provisional Application Ser. No. 63/370,411, filed Aug. 4, 2022, the complete disclosure of which is incorporated herein by reference for all purposes.

### FIELD

This description generally relates to antimicrobial formulations for use in gels and fluids and methods for decreasing the risk of bacterial colonization and biofilm on devices that in-dwell, penetrate and/or navigate in the body.

### BACKGROUND

The global health system is entering a new age where deadly strains of pathogens are emerging and evolving that are not addressable with existing drugs and infection control technologies. Many are calling this the post-antibiotic age of infection risk, as pathogens emerge and evolve that are not responsive to current antibiotics, creating complex, new infection prevention and control circumstances where new innovations are critically needed. Experts, including the U.S. Center for Disease Control, state that this rapidly growing problem could be a greater source of early mortality than current cancer cases unless new innovations are created to address this rapidly escalating problem

Hospital-acquired infections (HAIs), for example, are often caused by multidrug-resistant bacteria such as methicillin-resistant Staphylococcus aureus (MRSA) and Pseudomonas. HAIs are typically difficult to treat and add complications, such as prolonged hospital stays and death, to an already sick patient. Incidences of HAI are thought to be underreported due to the use of inefficient medical tracking systems and fear of potential organizational repercussions. One known risk for HAI is the use of indwelling catheters, such as central venous access devices (CVADs). For example, at least 70% of all nosocomial bloodstream infections occur in patients who have CVADs. This includes patients in intensive care, patients with cancer, and patients who are in need of hemodialysis or total parenteral nutrition.

There is evidence that colonized catheters can be important carriers of MRSA and other multidrug-resistant bacteria in communities and hospitals worldwide. The processes that can cause catheter-related complications include microbial colonization and biofilm formation. Biofilms have a specialized physiology where cells aggregate together and become encased in a self-produced polysaccharide and protein matrix that protects the cells from harsh environmental elements. Harsh conditions for microorganisms within an indwelling catheter would include mechanical flushing and the use of antibiotic agents. Each time the catheter is used, there is potential for resistant microorganisms sloughing off from a biofilm and entering the patient's bloodstream or body cavity.

These infections complicate the treatment of already sick individuals and cost the existing health care systems around the world millions of dollars. The microbes that colonize indwelling catheters and other devices typically form multicellular biofilms that are difficult to dislodge and are resistant to antimicrobial treatments. In addition, the colonization of these devices by microorganisms often precludes their long-term use, which can be a problem for human patients that have few body sites available for new catheters. The colonizing organisms often form biofilms, and increasingly these organisms are resistant to multiple antibiotics, making them difficult to treat.

A related infection issue exists with single-use disposable devices that must be placed in patients for a limited duration. These devices are exposed to external and internal bacteria, as well as varying temperature changes and other risk elements. Over time, these devices begin to support the colonization of bacteria and eventually biofilm growth, leading to the promotion of infection because of these indwelling devices. In addition, the devices can carry external pathogens into the body as they are placed in the patient. These devices include, by way of example but not limitation, Foley catheters, central venous catheters, arterial lines, drainage catheters, peripherally inserted central catheters, drug delivery ports, endotracheal tubes, and other devices that in-dwell, penetrate and/or navigate in the body.

For example, catheter-associated urinary tract infections account for over 1 million cases in the US alone and almost 80% of the nosocomial infections worldwide. Biofilms are a major problem faced by urinary catheter patients because of the inherent property of urine to deposit minerals once infection by any microbe has occurred. Free-floating, or planktonic, bacteria come across a surface submerged in the fluid and within minutes become attached. These attached bacteria produce slimy, extracellular polymeric substances (EPS) that colonize the surface and form the conditioning film. Extracellular polymeric substance production allows the emerging biofilm community to develop a complex, three-dimensional structure that is influenced by a variety of environmental factors. Biofilm communities develop within hours.

Catheter-associated urinary tract infection, when left untreated, may cause infections in the kidneys (pyelonephritis) and bloodstream (septicemia), leading to sepsis or, in extreme cases, even death. The urinary catheter, a partially implanted device, can cause a patient to be highly prone to infections mostly because of cross contamination from the drainage bag and the rich microbial flora in the skin. This susceptibility increases with the duration of catheterization, which allows bacteria to flourish.

A major hindrance in attacking and eliminating these biofilms is the extracellular polymeric substance that protects the cells, which allows the biofilm to exude high tolerance to stress from antibiotics and other biocidal treatments. In fact, a biofilm's tolerance to antibiotics has been attributed to three possible characteristics of the biofilm: 1) slow penetration of antibiotics due to the matrix formed by the exopolysaccharides; 2) formation of a resistant phenotype called persister cells that remain in a transient dormant state and can cause recurrent infections; and 3) an altered environment within the biofilm that is composed of different anaerobic niches, concentration gradients and local accumulation of acids and inhibitive waste products.

Infection control practitioners and catheter management teams worldwide have been aggressively searching for antimicrobial solutions to disinfect and keep indwelling catheters free of microorganisms. There is currently no approved solution that can effectively protect devices against this threat. General strategies for preventing bacteria formation include measures such as adherence to hand hygiene, limiting the use and duration of urinary catheters, using aseptic technique for catheter insertion, and adhering to proper catheter care.

Silver is one of the few antimicrobial agents for urinary catheter coatings (along with other medical devices) that is approved by the FDA. Even low concentrations of Ag ions are enough to kill microbes. However, silver-alloy coatings have been found to have less microbial efficacy than antibiotics. In addition, the strong oxidative activity of Ag-NPs releases silver ions, which results in several negative effects on biological systems by inducing cytotoxicity, genotoxicity, immunological responses, and even cell death

While antibiotics may seem to be a good alternative to the cytotoxicity problems caused by silver-alloy catheter coatings, the largest issue associated with antibiotics is the inherent problem of bacterial resistance, which can render these antibiotics useless after second or third applications. Antibiotic resistance has become more challenging since biofilms require higher doses of antibiotics and, in turn, the common infections caused by the bacteria lead to an increase in the resistance rate.

What is needed, therefore, are improved antimicrobial formulations and methods for decreasing the risk of bacterial colonization and biofilm on devices that in-dwell, penetrate and/or navigate in the body. It is particularly desirable to provide antimicrobial formulations that do not cause damage to the patient's cells or cause an increase in bacterial resistance

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

Antimicrobial formulations are provided for use in gels, fluids, medical swabs, cleaning wipes and the like and methods for killing pathogens and/or decreasing the risk of bacterial colonization and biofilm on devices that in-dwell, penetrate and/or navigate in the body. The formulations described herein may be particularly beneficial in both the prevention and treatment of biofilms found within, on, or near these devices, as well as biofilms found on, or within, body tissue, such as specimen collection sites and abiotic (i.e., wound dressing) and biotic (i.e., wound bed) environments.

In one aspect, an antibiotic gel formulation is provided that comprises ethylenediaminetetraacetic acid (EDTA) in about 1 percent to about 6 percent by weight. Applicant has discovered that the ability of EDTA to chelate and potentiate the cell walls of bacteria and destabilize biofilms by sequestering calcium, magnesium, zinc, and iron makes it a particularly useful agent for destroying biofilm, toxins, bacteria, prions, fungi, viruses or other pathogens.

In certain embodiments, the EDTA is present within the gel formulation in about 2 percent to about 4 percent by weight. Applicant has also discovered that this range of EDTA in the gel provides sufficient protection against biofilms and other pathogens, while inhibiting or completely preventing the adverse effects that may occur with higher percentages of EDTA, such as toxicity and/or blood thinning.

In certain embodiments, the EDTA comprises the tetrasodium form of EDTA, which is the salt resulting from the neutralization of EDTA with four equivalents of sodium hydroxide (or an equivalent sodium base).

The gel formulation may further comprise a lubricating gel, such as any suitable lubricant currently used in the field for catheter lubrication. The lubricating gel may, for example, comprise a water-soluble lubricant, a hydrophilic coating and/or silicone oil. The lubricant may comprise glycerin, hydroxyethycellulose, gluconolactone, methylparaben, sodium hydroxide, chlorhexidine gluconate or a combination of the above.

The gel formulation may comprise an anesthetic. The lubricant may comprise lidocaine, typically about 2 percent by weight. The gel formulation may comprise a surfactant.

In certain embodiments, the gel formulation further comprises ethanol. Applicant has discovered that EDTA and ethanol provide a synergistic anti-microbial formulation. In particular, ethanol destroys pathogens and biofilms almost immediately, whereas the EDTA has a longer-acting effect. This combination of EDTA and ethanol provides an anti-microbial formulation that quickly attacks existing pathogens and also provides longer term protection against the formation of biofilms. In certain embodiments, the ethanol is present within the gel formulation in about 1.5 percent to about 2 percent by weight.

In another aspect, methods are provided for decreasing the risk of bacterial colonization and biofilm on an endoscopic device configured for advancement through an opening into the patient. The endoscopic device may include, for example, an endoscope, trocar, cannula, dilatation device, biopsy brush, needle or forceps, Foley catheter, peripherally inserted central catheter (PICC), central venous access device (CVAD), a hemodialysis catheter, a tunneled or non-tunneled catheter, a cuffed or uncuffed catheter, a chest tube for pleural effusions, a peritoneal catheter for recurrent ascites, a guidewire, a stone retrieval device, a bipolar or monopolar electrosurgical or ultrasonic device, such as an ultrasound probe, a snare, an endoscopic stapler and other clamping or sealing instrument, an arterial line, drainage catheter, drug delivery port, endotracheal tube, implantable devices, such as electrical nerve stimulators, defibrillators, stents, pacemakers, joint implants, internal fixation devices, spinal implants and other devices that in-dwell, penetrate and/or navigate in the body.

In one embodiment, a method comprises impregnating a lubricating gel with ethylenediaminetetraacetic acid (EDTA), applying the lubricating gel to a distal portion of an endoscopic device, such as a catheter, and inserting the distal portion of the device into an opening in the patient. The EDTA-impregnated gel decreases the risk of bacterial colonization and biofilm on the distal portion of the device that enter and/or resides within the patient's body.

The endoscopic device may be inserted into the patient through a natural orifice (e.g., mouth, nose or anus) or through a percutaneous, endoscopic or open surgical procedure. In certain embodiments, the device is an in-dwelling catheter that is left in place within the patient for a period of time, such as a peripherally inserted central catheter (PICC) or a central venous access device (CVAD). The EDTA-impregnated gel protects the portion of the catheter within the patient from biofilm, toxins, bacteria, prions, fungi, viruses or other pathogens.

The lubricating gel may be impregnated with EDTA in about 1 percent to about 6 percent by weight, preferably in about 2 percent to about 4 percent by weight. The lubricating gel may comprise a water-soluble lubricant, a hydrophilic coating and/or silicone oil. The lubricant may comprise glycerin, hydroxyethycellulose, gluconolactone, methylparaben, sodium hydroxide, chlorhexidine gluconate or a combination of the above. The lubricant may comprise lidocaine, typically about 2 percent by weight.

In certain embodiments, the lubricating gel is also impregnated with ethanol, preferably in about 1.5 percent to about 2 percent by weight.

In embodiments, the method further comprises injecting a fluid into an internal lumen within the catheter. The fluid comprises saline or another suitable physiological water-based fluid and EDTA. The EDTA in the fluid prevents the formation of biofilms and or destroys pathogens residing within the catheter.

In another aspect, a guidewire is advanced through an internal lumen of the catheter. The guidewire may, for example, comprise an ultrasound probe at its distal tip. The EDTA-impregnated lubricating gel is applied to the guidewire.

In other embodiments, the method comprises injecting an anesthetic into the catheter access-site on the patient. The anesthetic comprises EDTA to prevent the formation of, or destroy, pathogens at the access site.

In another aspect, a method for inserting an indwelling catheter comprises providing a fluid, such as isotonic saline, that includes EDTA, injecting the fluid into an internal lumen of the catheter and inserting the catheter into an opening in the patient. The catheter may, for example be inserted the into a bladder of the patient, such as a urethral or suprapubic (e.g., Foley) catheter used to drain urine from the bladder. These catheters typically have a balloon at their distal end portion that secures the catheter within the bladder of the patient. Urine pools around the balloon, creating the potential for a catheter-associated urinary tract infection (CAUTI). Injecting saline and EDTA into this site inhibits or eliminates the risk of such infection from occurring.

In certain embodiments, a lubricating gel is provided that is impregnated with EDTA. The gel is applied to the catheter and/or the balloon.

In another aspect, a cleaning material is provided that comprises an absorbent material and a fluid solution at least partially saturating the absorbent material. The fluid solution comprises EDTA in about 1 percent to about 6 percent by weight, preferably about 2 to about 4 percent by weight.

The cleaning material may comprise a medical swab that includes an applicator for cleaning wounds, applying ointments, and/or taking specimens for analysis. The cleaning material may comprise a hospital wipe for sterilizing, cleaning or disinfecting a wound site, surgery site or other exposed area on a patient. The EDTA helps prevent bacterial infection at the site of application.

The fluid solution may comprise an antibiotic solution. The solution may further comprise ethanol, preferably in about 1.5 percent to about 2 percent by weight.

In another aspect, a kit is provided that includes an antibiotic gel formulation comprising EDTA in about 1 percent to about 6 percent by weight, a syringe comprising a needle coupled to a barrel with an internal chamber, and a plunger for ejecting contents from the internal chamber and a tube containing a fluid comprising saline and EDTA in about 1 percent to about 6 percent by weight. The kit is particularly useful for indwelling catheters. The gel formulation may be applied to the distal portion of the catheter and the saline and EDTA may be injected into one or more lumens of the catheter.

### BRIEF DESCRIPTION OF THE DRAWINGS

The accompanying drawings, which are incorporated in and constitute a part of this specification, illustrate several embodiments of the description and together with the description, serve to explain the principles of the description.

FIG. 1 illustrates an antibiotic gel formulation;

FIG. 2 illustrate an antibiotic kit;

FIG. 3 illustrates a medical swab containing the antibiotic gel formulation of FIG. 1;

FIG. 4 illustrates an indwelling catheter;

FIG. 5 illustrates a method for using the indwelling catheter of FIG. 4;

FIG. 6 illustrates a peripherally inserted central catheter (PICC) or central venous catheter;

FIG. 7 is an expanded view of the catheter of FIG. 6; and

FIG. 8 illustrates a catheter and a guidewire advancing through an internal lumen of the catheter.

### DETAILED DESCRIPTION OF THE PREFERRED EMBODIMENTS

Particular embodiments of the present description are described hereinbelow with reference to the accompanying drawings; however, it is to be understood that the disclosed embodiments are merely exemplary of the description and that the description may be embodied in various forms. Therefore, specific structural and functional details disclosed herein are not to be interpreted as limiting, but merely as a basis for the claims and as a representative basis for teaching one skilled in the art to variously employ the present description in virtually any appropriately detailed structure. Well-known functions or constructions are not described in detail to avoid obscuring the present description in any unnecessary detail. It should be understood also that the drawings are not drawn to scale and are not intended to represent absolute dimensions or relative size. Instead, the drawings help to illustrate the concepts described herein.

The present description provides antimicrobial formulations that include EDTA for use in gels, fluids, medical swabs, cleaning wipes, syringes and the like and kits including such antimicrobial formulations. Methods are also provided for destroying pathogens and/or decreasing the risk of bacterial colonization and biofilm on devices that in-dwell, penetrate and/or navigate in the body. The formations described herein may be particularly beneficial in both the prevention and treatment of biofilms found within, on, or near these devices, as well as biofilms found on, or within, body tissue, such sites for specimen collection (e.g., nose, ear, mouth, etc.), abiotic (i.e., wound dressing), biotic (i.e., wound bed) environments and the like.

Ethylenediaminetetraacetic acid (EDTA) is an aminopolycarboxylic acid with the formula: CH₂N(CH₂CO₂H)₂. This white, water-soluble solid is widely used to bind to iron and calcium ions. It binds these ions as a hexadentate (“six-toothed”) chelating agent EDTA is produced as several salts, notably disodium EDTA, sodium calcium edetate and tetrasodium ETA. EDTA is sometimes prescribed by doctors to clean toxic metals, such as lead, from the blood. Doctors have used the molecule for decades to treat heavy metal poisoning. In those cases it is given through an IV. EDTA is also an ingredient in some prescription cancer-fighting medicines. EDTA has been used for decades as a powerful anticoagulant preventing clot formation in vitro and is still being used in this capacity. However, the form of EDTA most often used is the disodium salt, which has reduced antimicrobial properties.

Applicant has discovered that the tetrasodium form of EDTA in a specific formulation has the ability to disrupt biofilms and destroy other pathogens, such as Gram-positive and Gram-negative bacteria, and certain fungal species. In particular, the ability of this form of EDTA to chelate and potentiate the cell walls of bacteria and destabilize biofilms by sequestering calcium, magnesium, zinc, and iron makes it a suitable agent for use in the management of biofilms.

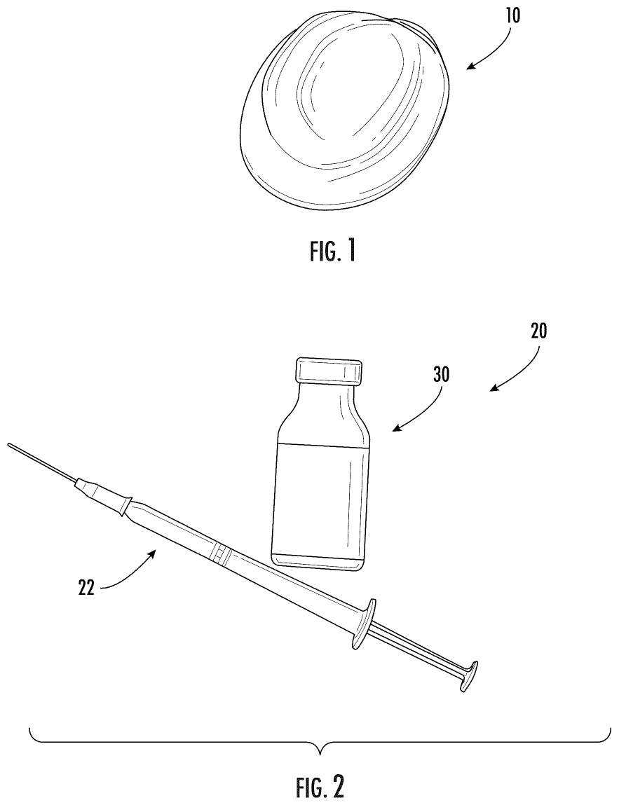

Referring now to FIG. 1, an antibiotic gel formulation 10 comprises EDTA in about 1 percent to about 6 percent by weight. In certain embodiments, the EDTA is present within the gel formulation in about 2 percent to about 4 percent by weight. The EDTA preferably comprises the tetrasodium form of EDTA, which is the salt resulting from the neutralization of EDTA with four equivalents of sodium hydroxide (or an equivalent sodium base).

Gel formulation 10 may further comprise a lubricating gel, such as any suitable lubricant currently used by those of skill in the art for catheter lubrication. The lubricating gel comprise, for example, a water-soluble lubricant, a hydrophilic coating and/or silicone oil. The lubricant may comprise glycerin, hydroxyethycellulose, gluconolactone, methylparaben, sodium hydroxide, chlorhexidine gluconate or a combination of the above (e.g., K-Y jelly).

Gel formulation 10 may comprise an anesthetic, such as lidocaine, mepivacaine, prilocaine, bupivacaine, etidocaine, and ropivacaine and levobupivacaine. In one embodiment, gel formulation 10 comprises lidocaine in about 2 percent by weight.

Gel formulation 10 may comprise a surfactant to lower the surface tension of formulation 10 and reduce the friction when it is applied to, for example, a medical device. Suitable surfactants for use with gel formulation 10 include nonionic, anionic, cationic and amphoteric surfactants.

In certain embodiments, gel formulation 10 further comprises ethanol. Applicant has discovered that EDTA and ethanol provide a synergistic anti-microbial formulation. In particular, ethanol destroys pathogens and biofilms almost immediately, whereas the EDTA has a longer-acting effect. This combination of EDTA and ethanol provides an anti-microbial formulation that quickly attacks existing pathogens and also provides longer term protection against the formation of biofilms. In certain embodiments, the ethanol is present within gel formulation 10 in about 1.5 percent to about 2 percent by weight.

Referring now to FIG. 2, a kit 20 is provided that includes gel formulation 10, a syringe 22 and a vial, ampule, bottle, tube or other container 30 of fluid. Syringe 22 comprises a needle coupled to a barrel with an internal chamber, and a plunger for ejecting contents from the internal chamber. Syringe 22 may include any syringe suitable for withdrawing the fluid from container 30 and injecting it into another location, such as a body tissue or within a medical device designed to enter the body.

Container 30 includes a fluid that can be safely used within a patient's body, such as isotonic saline or the like, and EDTA. The fluid preferably comprises the EDTA in about 1 percent to about 6 percent by weight. In certain embodiments, the EDTA is present within the gel formulation in about 2 percent to about 4 percent by weight. The EDTA preferably comprises the tetrasodium form of EDTA, which is the salt resulting from the neutralization of EDTA with four equivalents of sodium hydroxide (or an equivalent sodium base). In certain embodiments, the fluid further comprises ethanol in about 1.5 percent to about 2 percent by weight.

As described in detail below, kit 20 is particularly useful for indwelling catheters. Gel formulation 10 may be applied to the distal portion of the catheter and the fluid from container 30 may be injected into one or more lumens of the catheter.

Referring now to FIG. 3, a medical swab 50 is provided that includes an absorbent material 52, such as cotton or the like, coupled to an applicator 54. Medical swab 50 further comprises a fluid solution at least partially saturating absorbent material 52. The fluid solution comprises EDTA in about 1 percent to about 6 percent by weight, preferably about 2 to about 4 percent by weight.

Medical swab 50 is particularly useful for cleaning wounds, applying ointments, and/or taking specimens for analysis. The EDTA in the fluid solution helps prevent bacterial infection at the site of application.

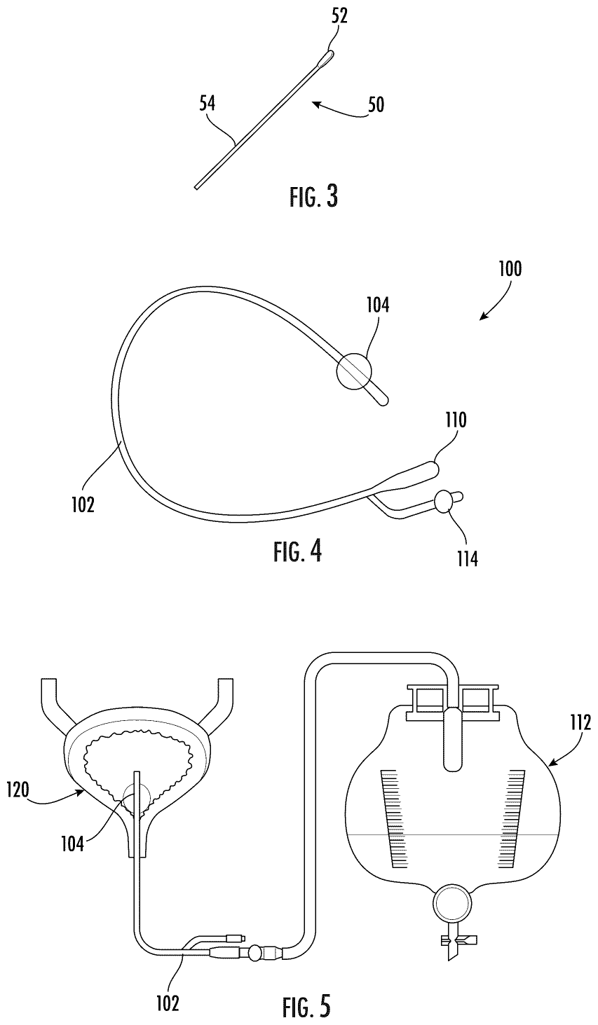

Referring now to FIGS. 4 and 5, a method for decreasing the risk of bacterial colonization and biofilm on an indwelling catheter will now be described. In this embodiment, a catheter 100 includes a tube 102 having at least one internal lumen (not shown) and a balloon 104 attached to the distal end of tube 102. In one embodiment, tube 102 has two separated channels, or lumens, running down its length. One lumen has an open distal end and a proximal end 110 configured for coupling to a collection bag 112. The other lumen has a valve 114 on its distal end and is connected to balloon 104.

As shown in FIG. 5, catheter 100 is a urethral or suprapubic (e.g., Foley) catheter used to drain urine from a patient's bladder 120. Balloon 104 holds tube 102 in place and urine drains through the second lumen in tube 102 into collection bag 112.

While these types of catheters 100 are particularly effective, they suffer from a few drawbacks. Urine pools around balloon 104, which cause free-floating, or planktonic, bacteria to come across the surface of the balloon and within minutes become attached. These attached bacteria produce slimy, extracellular polymeric substances (EPS) that colonize the surface and form a biofilm, resulting in a catheter-associated urinary tract infection (CAUTI).

According to the method described herein, a fluid solution is injected through the two internal lumens of catheter 100. The fluid solution includes saline and EDTA in one or the formulations described above. The fluid solution is preferably injected such that it resides around balloon 104 within the patient's bladder 120. The EDTA destroys pathogens contacting the surface of balloon and/or that are free floating within the pooled urine. In addition, the EDTA prevents the formation of biofilms in, or around the balloon.

In certain embodiments, a lubricating gel formation containing EDTA, such as those described above, is applied to at least the distal portion of tube 102. The lubricating gel is preferably designed to stick to tube 102 so that when tube is inserted into the patient's bladder, the gel provides a protective coating around tube 102, preventing biofilms from forming thereon. The lubricating gel may also be applied around the outer surface of balloon 104 to provide additional protection from such biofilms that may attempt to form on this outer surface.

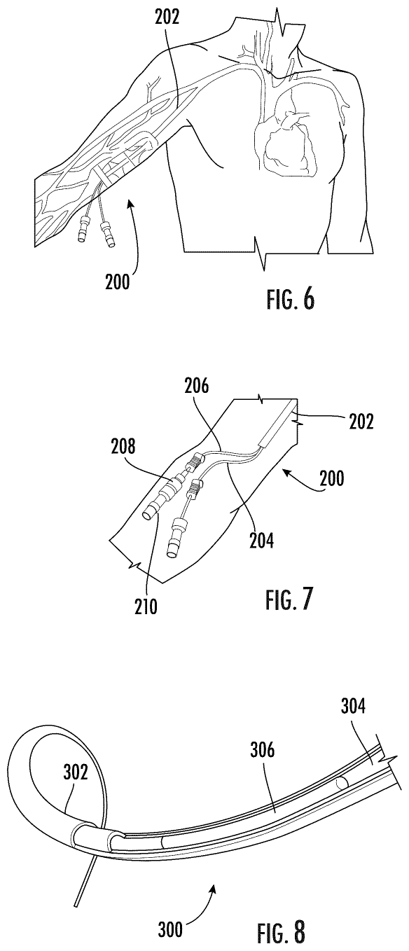

Referring now to FIGS. 6 and 7, a method will be described for decreasing the risk of bacterial colonization and biofilm on an indwelling catheter, such as a peripherally inserted central catheter (PICC), also called a PICC line, a peripheral IV line (PIV), a central venous catheter (CVC), a portacath, a central venous access device (CVAD) a hemodialysis catheter, a tunneled or non-tunneled catheter, a cuffed or uncuffed catheter, a chest tube for pleural effusions, a peritoneal catheter for recurrent ascites, or the like. As shown, in-dwelling catheter 200 comprises a tube 202 configured for insertion through a vein in patient's arm or leg and may also include two or more lumens 204, 206 extending distally from tube, each having needleless connector 208 and a disinfection cap 210. The tube 202 may be, for example, passed through to the larger veins near the heart. The catheter may be designed to provide access to the large central veins in the heart to give medications or liquid nutrition, take blood samples or give blood infusions.

Since the catheter 200 is typically intended for longer-term use within the patient, it can be a site for biofilm formation. According to one method, a fluid solution is injected through the internal lumens of catheter 200. The fluid solution includes saline and EDTA in one or the formulations described above. The fluid solution is preferably injected such that it passes through the lumens and beyond the distal tip of catheter. The EDTA destroys pathogens contacting the internal surface of the lumens and/or on the distal tip of catheter 200. In addition, the EDTA prevents the formation of biofilms in, or around catheter 200.

In certain embodiments, a lubricating gel formation containing EDTA, such as those described above, is applied to at least the distal portion of tube 202. The lubricating gel may also be applied to the two or more lumens 204, 206 extending distally from tube, as well as connector 208 and a disinfection cap 210, The lubricating gel is preferably designed to stick to tube 102 so that when tube is inserted into the patient's vein, the gel provides a protective coating around tube 102, preventing biofilms from forming thereon.

Referring now to FIG. 8, a representative method will be described for decreasing the risk of bacterial colonization and biofilm on an endoscopic device configured for advancement through an opening into the patient. The endoscopic device may include, for example, an endoscope, trocar, cannula, dilatation device, biopsy brush, needle or forceps, guidewire, stone retrieval device, a bipolar or monopolar electrosurgical or ultrasonic device, a snare, endoscopic stapler and other clamping or sealing instrument, an arterial line, drainage catheter, drug delivery port, endotracheal tube, implantable devices, such as electrical nerve stimulators, defibrillators, stents, pacemakers, joint implants, internal fixation devices, spinal implants and other devices that in-dwell, penetrate and/or navigate in the body.

For purposes of this description, an opening means natural orifice openings through any pre-existing, natural opening into the patient, such as the mouth, sinus, ear, urethra, vagina or anus, or any access port provided through a patient's skin into a body cavity, internal lumen (i.e., blood vessel), etc. or through incisions, and port-based openings in the patient's skin, cavity, skull, joint, or other medically indicated points of entry. The endoscopic device may also be configured to pass through a working or biopsy channel within an endoscope (i.e., through the same access port as the endoscope). Alternatively, the endoscopic device may be configured to pass through an opening that is separate from the endoscope access point.

In the example of FIG. 8, the endoscopic device includes a catheter 300 having an elongate flexible shaft 302 with one or more internal lumen(s) 304 for the passage of instruments, such as a guidewire 306. The catheter 300 may be designed to pass through the vascular system, through body lumens, such as the gastrointestinal tract, or into a body cavity of the patient. The catheter may further include a distal opening (not shown) that provides entry into the patient's body for instruments, guidewire 306, medications, fluids or the like.

According to one method, a fluid solution is injected through the internal lumen(s) 304 of catheter 300. The fluid solution includes saline and EDTA in one or the formulations described above. The fluid solution is preferably injected such that it passes through the lumens 304 and beyond the distal tip of catheter 300. The EDTA destroys pathogens contacting the internal surface of the lumens 304 and/or on the distal tip of catheter 200 and prevents the formation of biofilms in, or around catheter 200. The EDTA may also prevent biofilms from forming on the instruments passing through lumen(s) 304, such as guidewire 306.

In certain embodiments, a lubricating gel formation containing EDTA, such as those described above, is applied to at least the distal portion of catheter 300. The lubricating gel may also be applied to lumen(s) 304 extending through catheter 306, or to guidewire 306. The lubricating gel provides additional protection against the formation of biofilms on catheter 300 or any of the instruments or guidewires passing therethrough.

According to one specific embodiment, the endoscopic device may comprise an ultrasound probe. Ultrasound probes may be used for a variety of medical procedures. For example, ultrasound (IVUS) is a catheter-based diagnostic procedure used to view the inside of a coronary artery, providing a real-time view. IVUS shows the degree of narrowing or thickening (stenosis) of an artery by providing a visual image of the inside of the artery (the lumen) and the atheroma (membrane/cholesterol loaded white blood cells) that are hidden within the artery wall. Physicians typically use IVUS to image the lining of an artery in preparation for, during or to review the results of an angioplasty or atherectomy. It is also used in the placement of stents.

According to one method, a lubricating gel is applied to the ultrasound probe before it is inserted through catheter 300. The lubricating gel comprises EDTA in one of the formulations described above. The probe is then advanced through one of the internal lumen(s) 304 of catheter 300 to the target site in the patient, e.g., a coronary artery. The lubricating gel kills pathogens in and around the ultrasound probe and prevents the formation of biofilms thereon.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the description. Accordingly, the present description is intended to embrace all such alternatives, modifications, and variances. As well, one skilled in the art will appreciate further features and advantages of the present description based on the above-described embodiments. Accordingly, the present description is not to be limited by what has been particularly shown and described, except as indicated by the appended claims.

For example, in one aspect, an antibiotic gel formulation is provided comprising ethylenediaminetetraacetic acid (EDTA) in about 1 percent to about 6 percent by weight.

A second embodiment is the first embodiment, wherein the EDTA is present in about 2 percent to about 4 percent by weight.

A third embodiment is any combination of the first 2 embodiments, wherein the EDTA comprises tetrasodium EDTA.

A 4^{th} embodiment is any combination of the first 3 embodiments, further comprising a water-soluble lubricant.

A 5^{th} embodiment is any combination of the first 4 embodiments, further comprising an anesthetic.

A 6^{th} embodiment is any combination of the first 5 embodiments, further comprising about 2 percent lidocaine.

A 7^{th} embodiment is any combination of the first 6 embodiments, further comprising ethanol.

An 8^{th} embodiment is any combination of the first 7 embodiments, wherein the ethanol is present in about 1.5 percent to about 2 percent by weight.

In another aspect, a method for delivering a catheter into a patient is provided. The method comprises impregnating a lubricating gel with ethylenediaminetetraacetic acid (EDTA), applying the lubricating gel to a distal portion of the catheter, and inserting the distal portion of the catheter into an opening in the patient.

A second embodiment is the first embodiment further comprising impregnating the lubricating gel with the EDTA in about 1 percent to about 6 percent by weight.

A 3^{rd} embodiment is any combination of the first 2 embodiments, wherein the EDTA is present in about 2 percent to about 4 percent by weight.

A 4^{th} embodiment is any combination of the first 3 embodiments, wherein the lubricating gel is a water-soluble lubricant.

A 5^{th} embodiment is any combination of the first 4 embodiments, further comprising impregnating the lubricating gel with ethanol.

A 6^{th} embodiment is any combination of the first 5 embodiments, wherein the ethanol is present in about 1.5 percent to about 2 percent by weight.

A 7^{th} embodiment is any combination of the first 6 embodiments, further comprising injecting an anesthetic into the opening, wherein the anesthetic comprises EDTA.

An 8^{th} embodiment is any combination of the first 7 embodiments, further comprising providing a fluid comprising saline and EDTA and injecting the fluid into an internal lumen of the catheter

A 9^{th} embodiment is any combination of the first 8 embodiments, further comprising inserting the distal portion of the catheter into a vein of the patient.

A 10^{th} embodiment is any combination of the first 9 embodiments, wherein the catheter is a peripherally inserted central catheter (PICC).

An 11^{th} embodiment is any combination of the first 10 embodiments, wherein the catheter is a central venous access device (CVAD).

A 12^{th} embodiment is any combination of the first 11 embodiments, further comprising advancing a guidewire through an internal lumen in the catheter and applying the lubricating gel to at least a distal portion of the guidewire.

A 13^{th} embodiment is any combination of the first 12 embodiments, wherein the guidewire comprises an ultrasound probe.

In another aspect, a medical cleaning material is provided comprising an absorbent material and a fluid solution at least partially saturating the absorbent material, the fluid solution comprising EDTA in about 1 percent to about 6 percent by weight.

A second embodiment is the first embodiment, wherein the fluid solution comprises an antibiotic solution.

A 3^{rd} embodiment is any combination of the first 2 embodiments, wherein the EDTA is present in about 2 percent to about 4 percent by weight.

A 4^{th} embodiment is any combination of the first 3 embodiments, wherein the fluid solution comprises ethanol.

A 5^{th} embodiment is any combination of the first 4 embodiments, wherein the ethanol is present in about 1.5 percent to about 2 percent by weight.

A 6^{th} embodiment is any combination of the first 5 embodiments, wherein the absorbent material is attached to a tube and the cleaning material is a medical swab.

A 7^{th} embodiment is any combination of the first 6 embodiments, wherein the cleaning material is a wipe.

In another aspect, a method for inserting an indwelling catheter is provided. the method comprises providing a fluid comprising saline and EDTA, injecting the fluid into an internal lumen of the catheter and inserting the catheter into an opening in the patient.

A second embodiment is the first embodiment wherein the EDTA comprises about 1 percent to about 6 percent by weight of the fluid.

A third embodiment is any combination of the first 2 embodiments, wherein the EDTA comprises about 2 percent to about 4 percent by weight of the fluid.

A 4^{th} embodiment is any combination of the first 3 embodiments, wherein the fluid further comprises ethanol.

A 5^{th} embodiment is any combination of the first 4 embodiments, wherein the ethanol comprises about 1.5 percent to about 2% by weight of the fluid.

A 6^{th} embodiment is any combination of the first 5 embodiments, further comprising applying a lubricating gel to a distal portion of the catheter, wherein the lubricating gel comprises EDTA.

A 7^{th} embodiment is any combination of the first 6 embodiments, further comprising inserting the catheter into a bladder of the patient.

An 8^{th} embodiment is any combination of the first 7 embodiments, wherein the catheter further comprises a balloon at a distal portion of the catheter, the method further comprising applying a lubricating gel to an outer surface of the balloon, wherein the lubricating gel comprises EDTA.

A 9^{th} embodiment is any combination of the first 8 embodiments, further comprising injecting the fluid into the balloon.

In another aspect, a kit is provided that comprises an antibiotic gel formulation comprising EDTA in about 1 percent to about 6 percent by weight, a syringe comprising a needle coupled to a barrel with an internal chamber and a plunger for ejecting contents from the internal chamber and a tube containing a fluid comprising saline and EDTA in about 1 percent to about 6 percent by weight.

A second embodiment is the first embodiment, wherein the EDTA is present in the gel formulation and the fluid by about 2 percent to about 4 percent by weight.

A third embodiment is any combination of the first 2 embodiments, wherein the gel formulation further comprises a water-soluble lubricant.

A 4^{th} embodiment is any combination of the first 3 embodiments, wherein the gel formulation further comprises an anesthetic.

A 5^{th} embodiment is any combination of the first 4 embodiments, wherein the gel formulation further comprises about 2 percent lidocaine.

A 6^{th} embodiment is any combination of the first 5 embodiments, wherein the gel formulation and the fluid further comprise ethanol.

A 7^{th} embodiment is any combination of the first 6 embodiments, wherein the ethanol is present in about 1.5 percent to about 2 percent by weight.

What is claimed:

1. An antibiotic gel formulation comprising ethylenediaminetetraacetic acid (EDTA) in about 1 percent to about 6 percent by weight.

2. The gel formulation of claim 1, wherein the EDTA is present in about 2 percent to about 4 percent by weight.

3. The gel formulation of claim 1, wherein the EDTA comprises tetrasodium EDTA.

4. The gel formulation of claim 1, further comprising a water-soluble lubricant.

5. The gel formulation of claim 1, further comprising an anesthetic.

6. The gel formulation of claim 5, further comprising about 2 percent lidocaine.

7. The gel formulation of claim 1, further comprising ethanol, wherein the ethanol is present in about 1.5 percent to about 2 percent by weight.

8. A method for delivering a catheter into a patient, the method comprising:
impregnating a lubricating gel with ethylenediaminetetraacetic acid (EDTA);
applying the lubricating gel to a distal portion of the catheter; and
inserting the distal portion of the catheter into an opening in the patient.

9. The method of claim 8, further comprising impregnating the lubricating gel with the EDTA in about 1 percent to about 6 percent by weight.

10. The method of claim 8, wherein the EDTA is present in about 2 percent to about 4 percent by weight.

11. The method of claim 8, further comprising impregnating the lubricating gel with ethanol, wherein the ethanol is present in about 1.5 percent to about 2 percent by weight.

12. The method of claim 8, further comprising injecting an anesthetic into the opening, wherein the anesthetic comprises EDTA.

13. The method of claim 8, further comprising providing a fluid comprising saline and EDTA and injecting the fluid into an internal lumen of the catheter.

14. The method of claim 8, further comprising inserting the distal portion of the catheter into a vein of the patient.

15. A method for inserting an indwelling catheter, the method comprising:
providing a fluid comprising saline and EDTA;
injecting the fluid into an internal lumen of the catheter; and
inserting the catheter into an opening in the patient.

16. The method of claim 15, wherein the EDTA comprises about 1 percent to about 6 percent by weight of the fluid.

17. The method of claim 15, wherein the fluid further comprises ethanol, wherein the ethanol comprises about 1.5 percent to about 2% by weight of the fluid.

18. The method of claim 15, further comprising inserting the catheter into a bladder of the patient.

19. The method of claim 18, wherein the catheter further comprises a balloon at a distal portion of the catheter, the method further comprising applying a lubricating gel to an outer surface of the balloon, wherein the lubricating gel comprises EDTA.

20. The method of claim 19, further comprising injecting the fluid into the balloon.
